(19) United States
(12) Patent Application Publication
(10) Pub. No.: US 2020/0165228 A1
(43) Pub. Date: May 28, 2020

(54) A METHOD FOR PRODUCING POTASSIUM 1,1 -DINITRAMINO-5,5-BISTETRAZOLATE AND EXPLOSIVE COMPOSITIONS COMPRISING SAID SALT

(71) Applicant: DETNET SOUTH AFRICA (PTY) LTD, Johannesburg (ZA)

(72) Inventors: Elmar Lennox Muller, Centurion (ZA); Thomas M. Klapotke, Munchen (DE); Jorg Stierstorfer, Worthsee (DE); Marc F. Bolter, Munchen (DE); Maurus Volkl, Munchen (DE)

(73) Assignee: DETNET SOUTH AFRICA (PTY) LTD, Johannesburg (ZA)

(21) Appl. No.: 16/612,954

(22) PCT Filed: May 10, 2018
(86) PCT No.: PCT/ZA2018/050022

§ 371 Date: Nov. 12, 2019

(30) Foreign Application Priority Data
May 12, 2017 (ZA) 2017/03279

(51) Int. Cl. C07D 403/04; C06B 25/34; C06B 21/00; C06B 25/28

(52) U.S. Cl. CPC C07D 403/04; C06B 25/34; C06B 25/28; C06B 21/0016; C06B 21/0008

(57) ABSTRACT

A method of producing K₂DNABT wherein a biztetrazole intermediate is nitrated using a nitrating agent selected from the following: dinitronium disulphate; a mixture of nitric acid and sulfuric acid; a mixture of nitric acid and phosphorous pentoxide; and nitric acid with acetic anhydride.

### CROSS-REFERENCE TO RELATED APPLICATIONS

This application is a U.S. national stage application of International Application No. PCT/ZA2018/050022 entitled “A METHOD FOR PRODUCING POTASSIUM 1,1-DINITRAMINO-5,5-BISTETRAZOLATE AND EXPLOSIVE COMPOSITIONS COMPRISING SAID SALT”, which has an international filing date of 10 May 2018, and which claims priority to South African Patent Application No. 2017/03279, filed 12 May 2017.

### BACKGROUND OF THE INVENTION

The invention relates generally to a new method of manufacturing potassium 1,1-dinitramino-5,5-bistetrazolate (“K₂DNABT”) and to an explosive composition which includes K₂DNABT.

K₂DNABT was first synthesized in 2015 using a sophisticated synthetic process involving numerous steps as shown in FIG. 1 and as disclosed in Fischer et al “Potassium 1,1′-Dinitramino-5,5′-bistetrazolate: A Primary Explosive with Fast Detonation and High Initiation Power”. The process involves a nitrating step using an expensive nitration reagent namely, N₂O₅ which is not commercially available and must be prepared freshly by reacting NO₂ and ozone.

K₂DNABT, however, shows a sensitivity towards impact, friction and electrostatic discharge and, to facilitate its safe handling and commercial use the product must be desensitized.

It is an objective of the invention to provide a method of making K₂DNABT in a way that addresses the aforementioned shortcomings and to desensitize K₂DNABT to allow practicable, safe and reliable deposition of an explosive mixture thereof onto a heating element to function as an igniter of explosives.

### SUMMARY OF THE INVENTION

The invention provides a method of producing K₂DNABT which includes the steps of:
a. reacting dialkyl carbonate with hydrazine hydrate to produce alkyl carbazate;
b. reacting the alkyl carbazate with glyoxal to produce dialkyloxy carbonyl glyoxal bishydrazone;
c. halogenating the dialkyloxy carbonyl glyoxal bishydrazone with a halogenating agent to form halogenated bishydrazone;
d. azidation of the halogenated bishydrazone with an azide to produce diazido dialkyloxycarbonylglyoxal bishydrazone;
e. cyclization of the diazido dialkyloxycarbonylglyoxal bishydrazone with a ring closing electrophile reactant to produce bistetrazole intermediate;
f. deprotecting the bistetrazole intermediate with a nitrating agent to produce a nitramino intermediate; and
g. alkaline hydrolyses of the nitramino intermediate with potassium hydroxide to produce K₂DNABT;
   wherein the nitrating agent is selected from the following: a mixture of about 10:1 nitric acid and phosphorous pentoxide; and a mixture of nitric acid with acetic anhydride in a range of between 1:1 and 4:1.

Preferably, the nitrating agent is the 4:1 mixture of nitric acid with acetic anhydride.

Steps (a) and (b) may be combined in a first one-pot reaction step in which hydrazine hydrate reacts with dialkyl carbonate to form alkyl carbazate, and then glyoxal is added to produce dialkyloxy carbonyl glyoxal bishydrazone.

Steps (c) and (d) may be combined in a second one-pot reaction step in which the dialkyloxy carbonyl glyoxal bishydrazone is dissolved in a first solvent before the halogenating (step (c)) to form halogenated bishydrazone, and the azidation (step (d)) to form diazido dialkyloxycarbonylglyoxal bishydrazone.

In the step (e), the diazido dialkyloxycarbonylglyoxal bishydrazone may be dissolved in a second solvent before cyclization to produce bistetrazole intermediate.

Steps (d) and (e) may be combined in a second alternative one-pot reaction step in which halogenated bishydrazone is dissolved in a second solvent before the azidation (step (d)) to form the bistetrazole intermediate and the cyclization (step (e)) to form the nitramino intermediate.

The first solvent may be any of the following: DMF, DMSO, NMP, sulfolane, DMA, dioxane, water, EtOH, chloroform, MeOH, MeCN, THF, ethanol and water, and DMSO.

In the step (c) the halogenating agent may be N-chlorosuccinimide (NCS).

The azide in step (d) may be an earth metal azide, for example, sodium azide, lithium azide. Preferably, the azide is sodium azide.

The second solvent may be any of the following: DMF, ethanol, sulfolane, THF, MeCN, dioxane, chloroform. Preferably chloroform is used.

The ring closing electrophile may be selected from: HCl, SOCl₂, POCl₃, SO₂Cl₂, CO₂Cl₂, sulphuric acid and NaCl. Preferably, the electrophile is SOCl₂ or HCl. The HCl, preferably, is in a 37% concentration.

The steps (f) and (g) may be combined in a third one-pot reaction step in which the bistetrazole intermediate and the nitrating agent are added to produce a reaction mixture which is then added to a solution of potassium hydroxide to produce K₂DNABT.

The potassium hydroxide solution may be a 85 wt. % solution.

In the preparation of the nitrating agent of phosphorous pentoxide or nitric acid, phosphorous pentoxide may be added to nitric acid in a molar ratio 1:10 at a temperature in the range −15° C. to 5° C.

In the preparation of the nitrating agent of nitric acid with acetic anhydride, acetic anhydride may be added to nitric acid in a molar ratio between 1:3 and 1:4 at a temperature in the range −15° C. to 5° C.

In the step (a) the dialkyl carbonate may be dimethyl carbonate or diethyl carbonate. Preferably, diethyl carbonate is used.

In another aspect of the invention there is provided an explosive composition for use as an ignitable formulation which includes the following components in the following amounts:
a. K₂DNABT: 94 to 85 wt. %;
b. binder: 5 to 10 wt. %; and
c. nitrocellulose (NC): 1 to 5 wt. %

The binder may be graphite.

The invention also extends to a method of producing the explosive composition which includes the steps of:
a. dissolving K₂DNABT in a solvent to produce a K₂DNABT solution;
b. adding a binder to the K₂DNABT solution to produce a slurry;
c. evaporating the solvent to produce a K₂DNABT/binder mixture; and
d. adding an energetic binder to the mixture to produce the explosive composition.

The binder may be graphite dust.

The energetic binder may be ethanolic nitrocellulose (NC).

The method may include an additional step, after step (d), of drying the explosive composition with a nitrogen gas stream to increase the viscosity of the composition.

The invention extends to a composition for use as an explosive igniter which includes the following components in the following amounts:
a. K₂DNABT: 94 to 85 wt. %;
b. graphite: 5 to 10 wt. %; and
c. nitrocellulose (NC): 1 to 5 wt. %

The final explosive product contains no heavy metals, and increases human safety, and decreases adverse environmental effects. The explosive has a relatively high VOD due to the high nitrogen/oxygen content.

The desensitization of the explosive product facilitates handling by lowering friction and impact sensitivity and facilitates handling of the explosive product. The liquid nature of the formulation also facilitates automated deposition of the formulation onto a heating element, for use in an explosive igniter. In contrast, a primary explosive like lead azide becomes more sensitive when combined with additives.

### BRIEF DESCRIPTION OF THE DRAWINGS

The invention is further described by way of examples with reference to the accompanying drawings wherein;

FIG. 1 shows a current process for producing K₂DNABT;

FIGS. 2A and 2B show respective processes for producing K₂DNABT according to the invention;

FIGS. 3A and 3B illustrate a combination of step (a) and step (b) conducted in the processes in FIGS. 2A and 2B, respectively;

FIG. 4A depicts a step (c) (halogenating) combined with a step (d) (azidation);

FIG. 4B shows a step (c) (halogenating) which forms a part of the processes shown in FIGS. 2A and 2B respectively;

FIGS. 5A and 5B show a step (d) (azidation) which is included in the processes shown in FIGS. 2A and 2B, respectively.

FIGS. 6A and 6B show a step (e) (cyclization) which is included in the processes shown in FIGS. 2A and 2B respectively;

FIGS. 7A and 7B show a step (f) (nitrating) and a step (g) (hydrolysis) which form part of the processes shown in FIGS. 2A and 2B; and

FIG. 8 is a diagrammatical representation of a method of producing an explosive material to according to the invention.

### DETAILED DESCRIPTION OF THE PREFERRED EMBODIMENTS

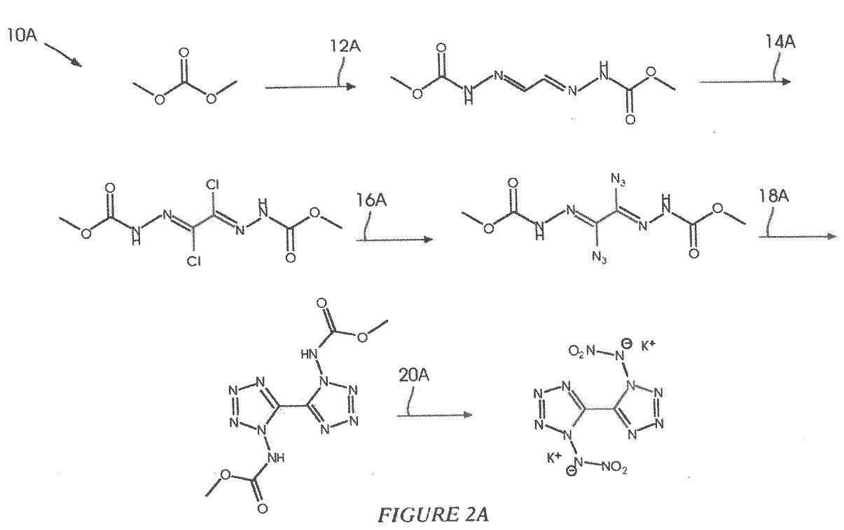

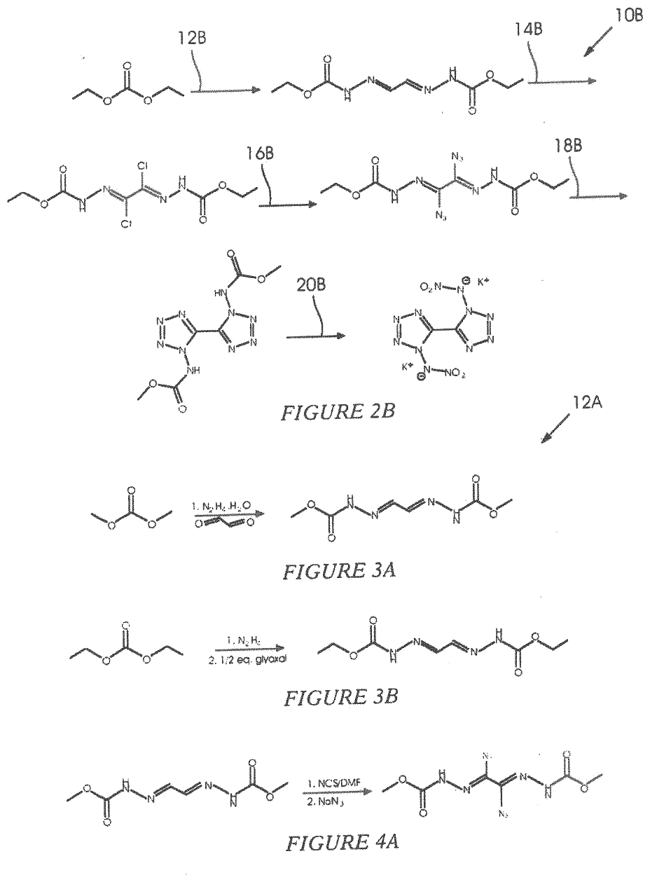

Methods 10A and 10B of producing K₂DNABT are schematically illustrated in FIGS. 2A and 2B, respectively. The difference between the methods shown in FIGS. 2A and 2B is as a result of a difference in the starting compounds, i.e. dimethyl carbonate and diethyl carbonate, respectively. The suffixes “A” and “B” are used to distinguish counterpart steps in the two methods.

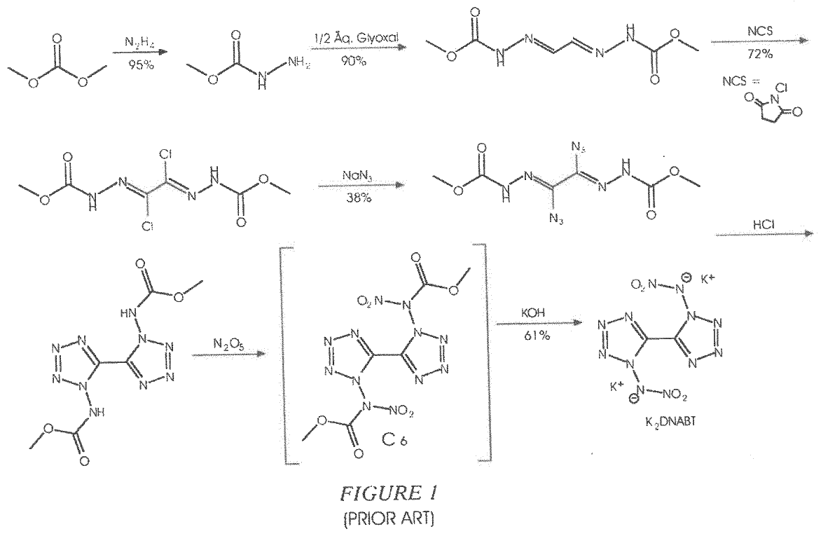

The method 10A, shown in FIG. 2A, comprises a step 12A wherein dimethyl carbonate is reacted with a hydrazine hydrate to produce a methyl carbazate intermediate which is then reacted with a glyoxal to produce a dimethoxy carbonyl glyoxal bishydrazone. The step 12A constitutes a combination, in a one-pot reaction, of the first two reaction steps of the original method shown in FIG. 1.

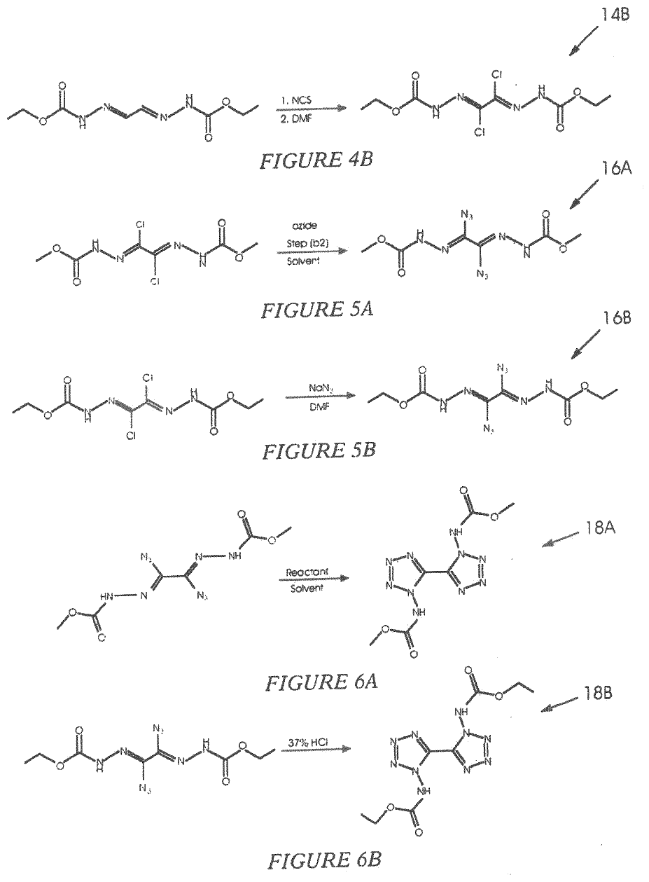

The dimethoxy carbonyl glyoxal bishydrazone is subjected to a halogenating step 14A wherein the dimethoxy carbonyl glyoxal bishydrazone is reacted with a halogenating agent, N-chlorosuccinimide (NCS), to form dichloro dimethoxy carbonyl glyoxal bishydrazone. This step is shown in FIG. 4A. Subsequently, as shown in FIG. 5A, dichloro dimethoxy carbonyl glyoxal bishydrazone is reacted with sodium azide in a step 16A (azidation) to form diazido-dimethoxy carbonyl glyoxal bishydrazone. Step 14A and step 16A could be combined in a single one-pot step to convert dimethoxy carbonyl glyoxal bishydrazone directly into diazido-dimethoxy carbonyl glyoxal bishydrazone without isolating dichloro dimethoxy carbonyl glyoxal bishydrazone.

In a step 18A (cyclization), diazido-dimethoxy carbonyl glyoxal bishydrazone is reacted with an electrophile, hydrochloric acid, to form dimethoxy carbonyl diaminobistetrazole as shown in FIG. 6A.

In a step 20A, dimethoxy carbonyl diaminobistetrazole is treated with a nitrating agent which comprises a mixture of nitric acid and acetic anhydride to form dimethoxy carbonyl dinitraminobistetrazole which is subsequently subjected to alkaline hydrolysis to form an end product, K₂DNABT.

FIG. 2B shows the method 10B which is similar to the method 10A. The method 10B utilizes, as a starting compound, diethyl carbonate. All subsequent compounds are accordingly based on ethyl compounds.

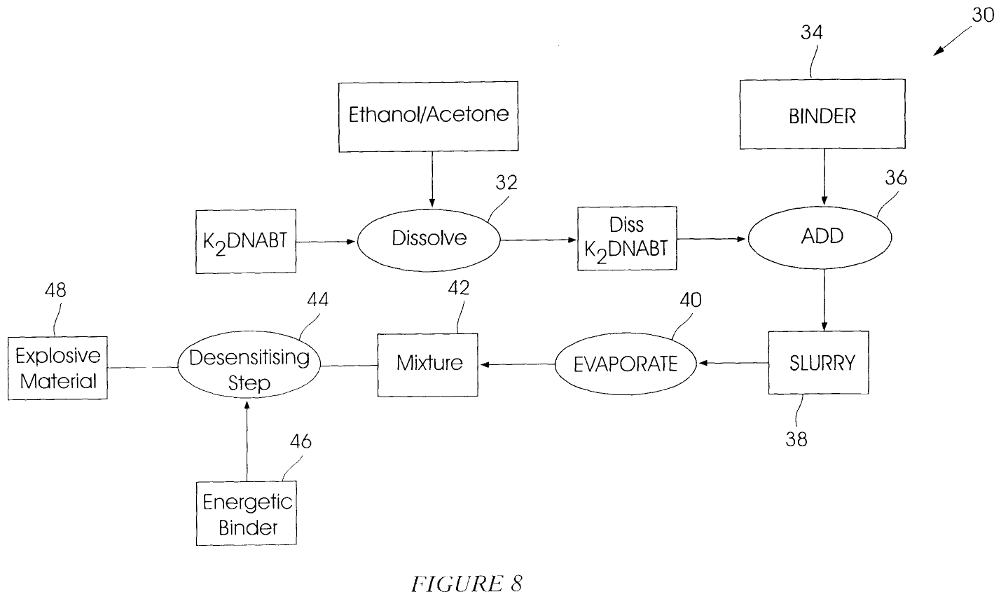

Overall the methods 10A and 10B have fewer synthesis steps than the method shown in FIG. 1. The reagents used are cheaper, the reaction conditions are milder. FIG. 8 is a diagrammatical representation of a method 30 of producing an explosive material. K₂DNABT, typically manufactured using the method 10A or 10B, is dissolved with ethanol or acetone in a dissolution step 32 to form a K₂DNABT solution. A binder 34 is added to the K₂DNABT solution in a step 36 to form a slurry 38. The solvent is evaporated in a step 40 to produce a K₂DNABT/binder mixture 42. In a desensitising step 44 an energetic binder 46 is added to the mixture 42 to produce an explosive composition 48.

### Procedure and Experimental Data (Method 10A)

In a step 12A, shown in FIG. 3A, (wherein step (a) and (b) are combined) dimethyl carbonate is treated with hydrazine hydrate and the resulting reaction mixture, containing a methyl carbazate intermediate, is stirred for one hour. Afterwards 500 mL of water is added to a glyoxal solution (40% in water). A nominal amount of acid (e.g. 37% of glacial acetic acid) is added to accelerate the precipitation of dimethoxy carbonyl glyoxal bishydrazone. The reaction is refluxed for at least one hour and stirred overnight. Dimethoxy carbonyl glyoxal bishydrazone is collected by filtration to achieve a 90% yield.

### Halogenating nd Azidation (Combined)

A feature of the current invention lies in the combined steps 14A and 16A, FIG. 4A. Prior to halogenating, dimethoxy carbonyl glyoxal bishydrazone is dissolved in dimethyl formamide (DMF) and N-chlorosuccinimide (NCS) (a halogenating agent) is added incrementally. After stirring at room temperature overnight, the reaction mixture is cooled to 0° C. and sodium azide is added. A resulting suspension is stirred overnight before ice water is added. The precipitate is collected by filtration giving a fairly low yield of 22% of diazido-dimethoxy carbonyl glyoxal bishydrazone.

The above protocol, redone with a larger amount of DMF, leads to a slightly better yield of diazido-dimethoxy carbonyl glyoxal bishydrazone at 28%.

An improvement in the yield of the chloro/azido exchange, which occurs with intermediate dichloro dimethoxy carbonyl glyoxal bishydrazone, was sought by using other solvents or azido compounds. As an alternative azido compound, lithium azide could be used as a replacement to sodium azide in step 16A.

Other solvents used in the pre-halogenating step were evaluated by suspending dimethoxy carbonyl glyoxal bishydrazone in each of a variety of solvents (see Table 1) to assess if yield improved. Sodium azide was added to each of the suspensions drop by drop. The reaction mixture was stirred overnight and triple the amount of water was added. The precipitate (diazido-dimethoxy carbonyl glyoxal bishydrazone) was collected by filtration and air dried.

TABLE 1

| | Dimethoxy | | | | Yield |
|---|---|---|---|---|---|
| | carbonyl glyoxal | | | | Diazido-dimethoxy |
| Reaction | bishydrazone | | Solvent in | | carbonyl glyoxal |
| number | in g | Solvent | mL | Temperature | bishydrazone |
| 1 | 1.0 | DMSO | 30 | r.t. | 40% |
| 2 | 2.0 | DMSO | 100 | r.t. | 49% |
| 3 | 10.0 | DMSO | 300 | r.t. | 65% |
| 4 | 1.0 | NMP | 15 | 0-5° C. | 29% |
| 5 | 2.0 | NMP | 100 | 0-5° C. | 19% |
| 6 | 1.0 | Sulfolane | 25 | r.t. | 0% (starting material) |
| 7 | 1.0 | DMA (Dimethyl | 25 | 0-5° C. | 30% |
| | | acetamide) | | | |
| 8 | 1.0 | Dioxane | 50 | r.t. | 5% |
| 9 | 1.0 | H₂O | 50 | r.t. | 10% |
| 10 | 1.0 | H₂O/DMSO 1:1 | 50 | r.t | 80% (starting material |
| | | | | | was also recovered) |

The highest yield obtained is with DMSO (65%), stirred overnight at room temperature. This is increased to an 80% yield with water/DSMO and the recovery of unreacted starting material.

Although DSMO produces the best yield, it is not a solvent of choice if step 14A and step 16A are combined in a one-pot reaction. Therefore a second series of tests was conducted with other solvents at temperatures between 35° C. and 100° C. (See Table 2).

TABLE 2

| | Dimethoxy | | | Yield |
|---|---|---|---|---|
| | carbonyl glyoxal | | | diazido-Dimethoxy |
| Reaction | bishydrazone | | | carbonyl glyoxal |
| number | in g | Solvent | Temperature | bishydrazone |
| 11 | 1.0 | Dioxane | 60° C. | 85% (dimethoxy carbonyl |
| | | | | glyoxal bishydrazone was |
| | | | | also recovered |
| 12 | 1.0 | Chloroform | 60° C. | 85% (dimethoxy carbonyl |
| | | | | glyoxal bishydrazone was |
| | | | | also recovered |
| 13 | 1.0 | EtOH | 35° C. | 30% |
| 14 | 1.0 | EtOH | 60° C. | 23% |
| 15 | 1.0 | EtOH | 100° C. | — |
| 16 | 1.0 | MeOH | 60° C. | 22% |
| 17 | 1.0 | MeCN | 60° C. | 10% (dimethoxy carbonyl |
| | | | | glyoxal bishydrazone was |
| | | | | also recovered |
| 18 | 1.0 | THF | 60° C. | |
| 19 | 1.0 | H₂O | 80° C. | 55% |
| 20 | 1.0 | H₂O | 60° C. | 48% |
| 21 | 1.0 | H₂O | 100° C. | 35% |
| 22 | 1.0 | H₂O | 1 h at100° C., 50° C. | 75% |
| | | | overnight | |
| 23 | 1.0 | EtOH/H₂O 1:1 | 60° C. | 33% |

This second series of tests resulted in improved yields of diazido-dimethoxy carbonyl glyoxal bishydrazone.

### Cyclization

There are different possibilities for successful ring closing of diazido-dimethoxy carbonyl glyoxal bishydrazone to form dimethoxy carbonyl diaminobistetrazole shown in FIG. 6A without using gaseous HCl, as in the prior art method of FIG. 1. Options are to use acetyl chloride (AcCl), trifluoroacetic acid (CF3—CO₂H), thionyl chloride (SOCl₂), phosphoryl chloride (POCl₃), sulfuryl chloride (SO₂Cl₂), oxalyl chloride (CO₂Cl₂), conc. hydrochloric acid (HCl) or sodium chloride (NaCl) as the electrophilic reactant (see Table 3).

TABLE 3

| | | | Amount | | | |
|---|---|---|---|---|---|---|
| Batch | Solvent | Reactant | (equivalents) | Result | Yield | Comment |
| 1 | Sulfolane | AcCl | | diazido-dimethoxy | — | r.t., 24 h |
| | | | | carbonyl glyoxal | | |
| | | | | bishydrazone | | |
| 2 | DMF | AcCl | | diazido-dimethoxy | — | r.t., 24 h |
| | | | | carbonyl glyoxal | | |
| | | | | bishydrazone | | |
| 3 | DMF | CF₃—CO₂H | | diazido-dimethoxy | — | r.t., 24 h |
| | | | | carbonyl glyoxal | | |
| | | | | bishydrazone | | |
| 4 | CHCl₃ | AcCl | | diazido-dimethoxy | — | r.t., 24 h |
| | | | | carbonyl glyoxal | | |
| | | | | bishydrazone | | |
| 5 | CHCl₃ | CF₃—CO₂H | | diazido-dimethoxy | — | r.t, 24 h |
| | | | | carbonyl glyoxal | | |
| | | | | bishydrazone | | |
| 6 | — | AcCl | | diazido-dimethoxy | — | r.t, 24 h |
| | | | | carbonyl glyoxal | | |
| | | | | bishydrazone | | |
| 7 | CHCl₃ | SOCl₂ | 8 | dimethoxy carbonyl | 90% | 55° C., 48 h |
| | | | | diaminobistetrazole | | |
| 8 | CHCl₃ | SOCl₂ (8 eq) + | | dimethoxy carbonyl | 70% | 55° C., 48 h |
| | | H₂O (2 mL) | | diaminobistetrazole | | |
| 9 | CHCl₃ | SOCl₂ | 16 | dimethoxy carbonyl | 34% | 55° C., 48 h |
| | | | | diaminobistetrazole | | |
| 10 | CHCl₃ | SOCl₂ | 10 | dimethoxy carbonyl | 37% | 55° C., 48 h |
| | | | | diaminobistetrazole | | |
| 11 | EtOH | SOCl₂ | 8 | dimethoxy carbonyl | 89% | 55° C., 48 h |
| | | | | diaminobistetrazole | | |
| 12 | THF | SOCl₂ | 8 | dimethoxy carbonyl | 77% | 55° C., 48 h |
| | | | | diaminobistetrazole | | |
| 13 | MeCN | SOCl₂ | 8 | dimethoxy carbonyl | 56% | 55° C., 48 h |
| | | | | diaminobistetrazole | | |
| 14 | Dioxane | SOCl₂ | 8 | dimethoxy carbonyl | 70% | 55° C., 24 h |
| | | | | diaminobistetrazole | | |
| 15 | CHCl₃ | POCl₃ | 4 | dimethoxy carbonyl | 72% | 55° C., 48 h |
| | | | | diaminobistetrazole | | |
| 16 | CHCl₃ | POCl₃ | 8 | dimethoxy carbonyl | 78% | 55° C., 48 h |
| | | | | diaminobistetrazole | | |
| 17 | CHCl₃ | SO₂Cl₂ | 8 | dimethoxy carbonyl | 82% | 55° C., 48 h |
| | | | | diaminobistetrazole | | |
| 18 | CHCl₃ | CO₂Cl₂ | 8 | diazido-dimethoxy | 82% | 55° C., 48 h |
| | | | | carbonyl glyoxal | | |
| | | | | bishydrazone and | | |
| | | | | dimethoxy carbonyl | | |
| | | | | diaminobistetrazole | | |
| 19 | 37% HCl | — | | dimethoxy carbonyl | 60% | 50° C., 24 h |
| | | | | diaminobistetrazole | | |
| 20 | conc. | NaCl | | dimethoxy carbonyl | 17% | r.t., 24 h |
| | H₂SO₄ | | | diaminobistetrazole | | |

Sulfolane, CHCl₃, DMF, EtOH, amongst others, were tried as alternative solvents for the ring closing reaction to form dimethoxy carbonyl diaminobistetrazole.

In one example, the diazido-dimethoxy carbonyl glyoxal bishydrazone was suspended in chloroform and thionyl chloride was added. The mixture was heated to 55° C. for 48 hours. The dimethoxy carbonyl diaminobistetrazole was collected by suction filtration. However, ring closing worked best (yield: 90% after recrystallization) by adding 8.0 equivalents of SOCl₂ (see Table 3).

Using EtOH, THF, dioxane and MeCN as alternative solvents, and SOCl₂, and SO₂Cl₂, cyclization occurred with high yield.

Successful ring closure of diazido-dimethoxy carbonyl glyoxal bishydrazone with high yields of dimethoxy carbonyl diaminobistetrazole was also achieved using POCl₃ and SO₂Cl₂ in chloroform—see batches 15-17 in the above Table 3.

In a preferable example, to achieve ring closure, diazido-dimethoxy carbonyl glyoxal bishydrazone is suspended in 37% HCl and heated overnight at 50° C. (batch 19). The product (dimethoxy carbonyl diaminobistetrazole) is clean and the yield is about 60%, which can be increased by a longer reaction time.

In another example, diazido-dimethoxy carbonyl glyoxal bishydrazone is suspended in sulfuric acid and sodium chloride is added incrementally. The mixture is stirred overnight at room temperature with water added. Dimethoxy carbonyl diaminobistetrazole is extracted with an organic solvent and a resulting mixture is then treated with ethyl acetate to remove the solvent and recover dimethoxy carbonyl diaminobistetrazole. The NMR spectra showed successful ring closing but with a low yield (17%) and residual starting material.

### Alternative: Azidation and Cyclization (Combined)

Alternatively, steps 16A and 18A can be combined in a one-pot reaction, without isolating the diazido-dimethoxy carbonyl glyoxal bishydrazone. Dichloro dimethoxy carbonyl glyoxal bishydrazone is used as a starting material and dimethoxy carbonyl diaminobistetrazole is isolated as an intermediate product.

For safety considerations, this one-pot reaction step is preferential.

The one-pot reaction step was tried with four different solvents: chloroform, ethanol, DMSO and DMF (see Table 4).

In each instance, dichloro dimethoxy carbonyl glyoxal bishydrazone is suspended in the chosen solvent and sodium azide is added at room temperature. The suspension is stirred overnight and SOCl2 is added. The reaction is heated at 55° C. for 2 days. In the case of ethanol, DMF and chloroform the solvent was removed in vacuo and the residue recrystallized in hot methanol. By using DMSO the mixture is diluted with water (150 mL) and extracted with EtOAc. The combined organic phases are dried over MgSO₄ and the solvent removed.

TABLE 4

| | | T in ° C. of | Yield/result |
|---|---|---|---|
| Reaction | | chloro/azide | dimethoxy carbonyl |
| number | Solvent | exchange | diaminobistetrazole |
| 1 | CHCl₃ | r.t. | 0% (chloro |
| | | | compound) |
| 2 | EtOH | r.t. | 0% (chloro |
| | | | compound) |
| 3 | DMSO | r.t. | 10% product |
| 4 | DMSO | 50° C. | — |
| 5 | DMF | r.t. | 0% (chloro |
| | | | compound) |
| 6 | H₂O; 37% | 80° C. (exchange); | 24% |
| | HCl | 50° C. (rina closina) | |

From Table 4 it can be seen that the one-pot reaction step worked with DMSO (batch 3) and conc. HCl (batch 6). The challenge with DMSO is getting rid of the solvent which is achieved by extensive extractions with EtOAc.

The preferred example, in the one-pot step, is with HCl with a yield of 24%. This step is easy and includes cheap and readily available reagents. However, the yield is low and needs to be improved. Yield improvement may be achieved by a longer reaction time for the chloro/azido exchange or use of less concentrated hydrochloric acid.

The one-pot step is to be contrasted with a two-step process (step (b₂) and step (c)) which yields of 60% for dimethoxy carbonyl diaminobistetrazole.

### Nitration

The scheme showed in FIG. 1 teaches nitrating dimethoxy carbonyl diaminobistetrazole to form dimethoxy carbonyl dinitraminobistetrazole by adding this compound dropwise to N₂O₅, (dissolved in MeCN at −5° C.) and quenching the solution by adding ice water to isolate an intermediate compound i.e. dimethoxy carbonyl dinitraminobistetrazole.

Dimethoxy carbonyl dinitraminobistetrazole has to be handled with care due to its sensitive behaviour towards impact (IS), friction (FS) and electrostatic discharge (ESD). Its sensitivity is similar to that of K₂DNABT (see Table 5).

TABLE 5

| | Diazido- | | | |
|---|---|---|---|---|
| | dimethoxy | Dimethoxy | Dimethoxy | |
| | carbonyl | carbonyl | carbonyl | |
| | glyoxal | diaminobis- | dinitraminobis- | |
| | bishydrazone | tetrazole | tetrazole | K₂DNABT |
| IS [J] | 2 | 3 | 1 | 1 |
| FS [N] | 18 | 192 | <5 | <5 |
| ESD [J] | 0.03 | 0.5 | 0.03 | 0.003 |

K₂DNABT is prepared from dimethoxy carbonyl dinitraminobistetrazole by the alkaline hydrolysis of the protecting groups using a 2M potassium hydroxide solution.

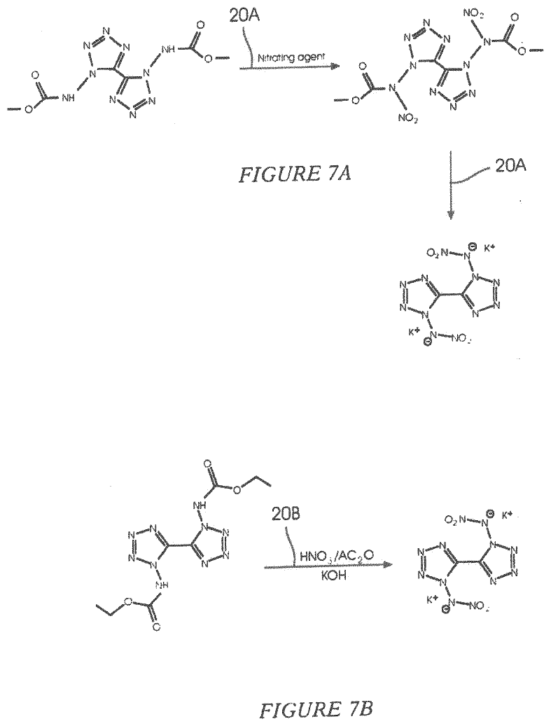

The disadvantage with the prior art method (FIG. 1) is that N₂O₂ is prepared from dinitrogen pentoxide in dry acetonitrile which is commercially unavailable. The preparation is laborious and includes expensive reagents. This is the motivation for a different nitration step 20A in the nitration of dimethoxy carbonyl diaminobistetrazole shown in FIG. 7A.

An alternative nitrating agent is selected from nitric acid, dinitronium disulphate ((NO₂)₂S₂O₇), mixed acid (HNO₃/H₂SO₄), nitric acid with phosphorous pentoxide (HNO₃/P₄O₁₀) and nitric acid with acetic anhydride (HNO₃/Ac₂O). The dinitronium disulphate is prepared according to the method of Ingold et al, J. Org. Chem., 1950 using N₂O₅ and SO₃.

TABLE 6

| Nitrating agent | Reaction conditions | Yield [%] |
|---|---|---|
| Nitric acid 100% | −10° C., 4 h | 0 |
| Dinitrogen pentoxide 1.3 | −10° C., 4 h | 0 |
| equivalents | | |
| Dinitrogen pentoxide 2.2 | −10° C., 2 h | 91% |
| equivalents | | |
| Dinitronium disulfate 2.2 | −10° C., 2-4 h | 19% |
| equivalents | | |
| Nitric acid 100%/P₄O₁₀ | 0° C., 6 h (new method) | 43% |
| Nitric acid 100%/Acetic | −10° C., 3 h | 91% |
| anhydride (4:1) | | |
| Nitric acid 100%/Acetic | −10° C., 3 h | 63% |
| anhydride (1:1) | | |
| Nitric acid 100%/Sulfuric acid | −10° C., 4 h | 0% |
| 96% | | |
| Acetyl nitrate (in MeCN), 16 | −10° C., 4 h | 0% |
| eq. | | |

Dinitronium disulphate replaces N₂O₅. This particular nitration agent is very similar to N₂O₅ and hydrolyses to one equivalent nitric acid and two equivalents of sulfuric acid upon contact with water.

In one example, dinitronium disulphate is dissolved in dry acetonitrile at 0° C. and dimethoxy carbonyl diaminobistetrazole is added. After 3 hours, a 2 M potassium hydroxide solution is added. Against all expectations, a two-phase system was obtained consisting of two solutions. Water was added until the liquid phases combined. Stirring is stopped and the solution was cooled to 0° C. After 1 hour no precipitate had formed and the synthesis attempt was a failure.

The experimental routine was repeated using 2.2 equivalents of dinitronium disulphate and the dimethyl carbonate of the reaction was doubled. This time, K₂DNABT could be obtained with an unexpectedly low yield of only 19%.

Thus, (NO₂)₂S₂O₇ is unsuitable for the preparation of K₂DNABT. The neutralization of 2.2 equivalents dinitronium disulphate requires excessive amounts of potassium hydroxide (2 M solution) due to the production of “mixed acid” upon contact with aqueous solutions. Large quantities of potassium sulphate are formed which is only slightly soluble at 0° C. and is less soluble than potassium nitrate. Hence, the removal of potassium sulphate requires large amounts of water. This results in the dissolution of most of the produced K₂DNABT. This causes a decrease in the yield.

In another example, mixed acid comprising 1 part 100% nitric acid and 2 parts 100% sulfuric acid, is cooled to −10° C. and dimethoxy carbonyl diaminobistetrazole is added. The resulting suspension is stirred for 4 hours at an initial temperature and subsequently poured into an ice-cold solution of 85 wt.-% potassium hydroxide, comprising a necessary amount of water for the complete dissolution of formed potassium nitrate and potassium sulphate. A large amount of precipitate was formed which dissolved almost completely upon mechanical stirring. The solid material is collected by suction filtration

Setting the filter paper alight gave a loud report. This is an indication that K₂DNABT is produced.

The problem with this example is that the amount of water needed for the complete dissolution of the inorganic by-products causes dissolution of K₂DNABT.

The preceding example is repeated using more starting material dimethoxy carbonyl diaminobistetrazole (0.50 g, 1.76 mmol) in comparison to the amount of mixed acid (nitric acid: 14.1 mmol, 0.88 g, sulfuric acid: 28.2 mmol, 2.66 g). The work-up routine is performed in the same manner as described above. The results were the same and no solid residue could be collected after the suction filtration.

A further example uses dinitrogen pentoxide which is generated in situ by the reaction of 100% nitric acid with phosphorus pentoxide. Here, phosphorus pentoxide (0.80 g, 2.82 mmol) is added to nitric acid (1.78 g, 28.2 mmol) at 0° C. using an ice-bath. Dimethoxy carbonyl diaminobistetrazole (0.25 g, 0.88 mmol) is added to the resulting slurry and mechanically stirred for 6 hours at an initial temperature. The reaction mixture is then poured into an ice-cold solution of 85 wt.-% potassium hydroxide (4.09 g, 62.0 mmol) comprising an amount of water for the complete dissolution of formed potassium nitrate and potassium phosphate. The resulting suspension is stirred at 0° C. for 30 minutes and the remaining solid is collected using suction filtration to give K₂DNABT (0.12 g, 0.36 mmol) with a yield of 43% without isolating dimethoxy carbonyl dinitraminobistetrazole.

The viscosity of the HNO₃/P₄O₁₀ mixture hinders diffusion in the reaction mixture. Therefore, longer reaction times may be needed for higher yields.

Several mixtures of 100% nitric acid and acetic anhydride with varying ratios of the reactants were tested in the step 20A of dimethoxy carbonyl diaminobistetrazole.

It is considered that dinitrogen pentoxide causes the nitration which is formed in situ as follows:

HNO₃+AcONO₂≥AcOH+N₂O₅

After a screening of the various nitration agents, the following nitrating step 18A is preferred as it offers the highest yield and purity of K₂DNABT.

Nitric acid (7.09 g, 0.11 mol) is cooled to −10° C. in a 25 mL round bottom flask using an ice bath. Acetic anhydride (2.84 mL, 30.03 mmol) is slowly added keeping the temperature below 0° C. Dimethoxy carbonyl diaminobistetrazole (1.00 g, 3.52 mmol) is added in small portions over a period of 10 minutes. After a reaction time of 1 hour a yellow solution is formed which turns into a yellowish suspension about 20 minutes later. After an overall reaction time of about 3 hours, the suspension is added to a solution of about 85% potassium hydroxide (11.40 g, 172.7 mmol) in 92.00 g of a 50:50 ice-water-mixture. Additional potassium hydroxide is added until pH 12 or higher is reached. A precipitate is formed which is collected by suction filtration, the precipitate is then washed with 2 mL of cold water and dried to yield 1.07 g (91° A) of K₂DNABT without isolating dimethoxy carbonyl dinitraminobistetrazole.

### Procedure and Experimental Data (Method 10B)

The method 10A shown in FIG. 2A involves a methylester protecting group. This is as a result of the dimethyl carbonate starting compound. Because of the poor solubility of the intermediate compounds and low yields in the chloro-azido exchange step 16A, an alternative method is proposed using a diethoxy protecting group. The essential difference between the prior method and the proposed method is the usage of diethyl carbonate instead of dimethyl carbonate as a starting reagent. An economic benefit is that diethyl carbonate is of lower cost compared to methyl carbonate.

Following the preferred steps described above, but with a diethoxyl carbonate starting reagent, K₂DNABT is successfully synthesized in this ethoxy group synthesis as shown in FIG. 2B. Table 7 shows that the yields of intermediate and end products are higher with the ethoxyl.

TABLE 7

| | | Yield | Yield |
|---|---|---|---|
| Compound | Step | Methoxy | Ethoxy |
| dimethoxy carbonyl | (b) | 90% | 84% |
| glyoxal bishydrazone/ | | | |
| diethoxy carbonyl | | | |
| glyoxal bishydrazone | | | |
| dichloro dimethoxy | (c) | 72% | 67% |
| carbonyl glyoxal | | | |
| bishydrazone/dichloro | | | |
| diethoxy carbonyl | | | |
| glyoxal bishydrazone | | | |
| diazido-dimethoxy | (d) | 60% in DMSO | 83% in DMSO |
| carbonyl glyoxal | | 75% H₂O | 65% H₂O |
| bishydrazone/diazido- | | | |
| diethoxy carbonyl | | | |
| glyoxal bishydrazone | | | |
| dimethoxy carbonyl | (e) | 60% | 65% |
| diaminobistetrazole/ | | | |
| diethoxy carbonyl | | | |
| diaminobistetrazole | | | |
| K₂DNABT | (f) | 61% (N₂O₅) | 83% (HNO₃/Ac₂O) |
| Total | | 14% DMSO | 25% DMSO |
| | | 18% H₂O | 20% H₂O |

Of interest are the sensitivity values (Table 8) of the azido compounds and ring closed compounds.

TABLE 8

| | Sensitivities methoxy | Sensitivities ethoxy |
|---|---|---|
| diazido-dimethoxy | IS: 2 J; FS: 18 N; | IS: 6 J; FS: 360 N; |
| carbonyl glyoxal | ESD: 0.03 J | ESD: 0.05 J |
| bishydrazone/diazido- | | |
| diethoxy carbonyl | | |
| glyoxal bishydrazone | | |
| dimethoxy carbonyl | IS: 3 J; FS: 192 N; | IS: 3 J; FS: 192 N; |
| diaminobistetrazole/ | ESD: 0.5 J | ESD: 0.65 J |
| diethoxy carbonyl | | |
| diaminobistetrazole | | |

The sensitivity value of the diazido-dimethoxy carbonyl glyoxal bishydrazone and diazido-diethoxy carbonyl glyoxal bishydrazone differ and this is due to the protecting group. Consequently, the diazido-diethoxy carbonyl glyoxal bishydrazone is less sensitive towards friction and impact. Thus, it is easier and safer to handle. Dimethoxy carbonyl diaminobistetrazole and diethoxy carbonyl diaminobistetrazole have similar sensitivity values.

Therefore, the method 10B is preferred to the method 10A because of better yields, a less sensitive intermediate diethoxy carbonyl diaminobistetrazole, lower cost starting materials and better solubility of products.

The step 12B in the production of diethoxy carbonyl glyoxal bishydrazone (a combination of steps (a) and (b)) is illustrated in FIG. 3B. An amount of 8.8 g (157 mmol) of hydrazine hydrate is added incrementally to 20.06 g (169 mmol) of diethyl carbonate, to form a ethyl carbazate intermediate, at room temperature. The mixture containing a ethyl carbazate intermediate is stirred for 3 hours at room temperature until homogenous. Subsequently, 300 mL of a water/ethanol mixture (1:1) and 11.2 g (77.2 mmol, 40% in H₂O) of glyoxal solution is added. To speed up the precipitation, 4 mL of conc. HCl (37%) is added and the mixture is stirred overnight. The precipitate is collected by filtration and washed with water, ethanol and ether to give diethoxy carbonyl glyoxal bishydrazone (15.1 g, 65.2 mmol, 85%), a slightly yellow solid.

### Halogenating

The step 14B in the production of dichloro diethoxy carbonyl glyoxal bishydrazone is illustrated in FIG. 4B. An amount of 5.0 g (21.74 mmol) of diethoxy carbonyl glyoxal bishydrazone is suspended in 100 mL dimethylformamide and 8.7 g (65.22 mmol, 3.0 eq.) of N-chlorosuccinimide (NCS) is added incrementally to the suspension. The reaction mixture is stirred overnight at room temperature. A resulting solid phase is then filtered off and washed with ethanol and diethylether to yield dichloro diethoxy carbonyl glyoxal bishydrazone (3.58 g, 12.0 mmol, 55%).

### Azidation

The step 16B in the production of diazido-diethoxy carbonyl glyoxal bishydrazone is illustrated in FIG. 5B. An amount 1.0 g (3.35 mmol) of dichloro diethoxy carbonyl glyoxal bishydrazone is suspended in 50 mL DMF and 0.5 g (7.4 mmol, 2.2 eq.) Sodium azide is added at 10° C. The mixture is stirred overnight at room temperature and is diluted with 100 mL of ice-water. The precipitate is collected by filtration and washed with water, ethanol and diethyl ether to yield diazido-diethoxy carbonyl glyoxal bishydrazone (0.4 g, 1.28 mmol, 40%).

### Cyclization

FIG. 6B illustrates the step 18B in the production of diethoxy carbonyl diaminobistetrazole. An amount of 0.4 g (1.28 mmol) of Glib diazido-diethoxy carbonyl glyoxal bishydrazone is suspended in 150 mL 37% HCl and heated overnight at 50° C. The resulting solution is extracted with ether (3×50 mL) and the solvent removed in a vacuum to give diethoxy carbonyl diaminobistetrazole (0.23 g, 0.74 mmol, 58%), a colourless crystal.

### Nitrating

In a final step 20B, illustrated in FIG. 7B, K₂DNABT is produced. An amount of 0.55 g (1.76 mmol) of diethoxy carbonyl diaminobistetrazole is suspended in HNO₃ (2.35 mL, 56.3 mmol, 100%) and cooled to −10° C. Within an hour Ac₂O (1.4 mL, 14.81 mmol) is added at −5° C. The resulting mixture is stirred between −5° C. and −10° C. for 3 hours. Subsequently, the mixture is added to an ice cold KOH solution (45 g ice, 6.0 g KOH). After stirring for 30 minutes the precipitate is collected by filtration giving K₂DNABT (0.49 g, 1.46 mmol, 83%), a colourless solid, without the isolation of diethoxy carbonyl dinitraminobistetrazole.

### Desensitization of K₂DNABT

Desensitization of the K₂DNABT is necessary. Wax, graphite and silicone oil have been tested as possible desensitization agents. Experimentally, different mixtures of the respective desensitizers were added to K₂DNABT and the corresponding sensitivities of the mixtures were determined (see Table 9).

TABLE 9

| measured value | 10 wt.-% | 20 wt.-% | 30 wt.-% |
|---|---|---|---|
| | wax | | |
| Impact (J) | <1 | <1 | <1 |
| Friction (N) | <5 | <5 | <5 |
| ESD (mJ) | 2 | 2 | 3 |
| | silicone oil 10.000 cSt | | |
| Impact (J) | 1.5-2 | 5 | 5 |
| Friction (N) | <5 | <5 | <5 |
| ESD (mJ) | 3 | 3 | 4 |
| | graphite flakes 10 microns | | |
| Impact (J) | 7 | >10 | >10 |
| Friction (N) | <5 | <5 | 5 |
| ESD (mJ) | 3 | 3 | 10 |

The best result was achieved using graphite dust. A mixture of 30 wt.-% graphite causes a drastic decrease of the ESD sensitivity, but a mixture of 10 wt.-% graphite offers the best overlap of a decreased sensitivity and a low mixture of non-energetic material.

The mixture of K₂DNABT and graphite is designated K₂DNABT-G.

The loss of performance due to the mixture is estimated using the EXPLO5-code. The calculated value for K₂DNABT-G (10% mixture):

Vdet.=8137 m/s, pCJ=26.97 GPa

A preferred method of desensitization is described below.

In a first step, K₂DNABT (200 mg) is added to a plastic test tube and covered with 2 mL ethanol or acetone. Graphite dust (22.2 mg) is added and mechanical stirred for 2 hours. The stirring was stopped and the solvent is evaporated using a nitrogen gas stream.

In a second step, an energetic binder is applied to K₂DNABT-G. K₂DNABT-G is left in the plastic test tube and a pre-calculated amount of a 1 wt.-% (2468.9 mg) ethanolic nitrocellulose (NC) solution is added in order to achieve a mixture of 10 wt.-% binder. The resulting suspension is dried using a nitrogen gas stream until a viscous, sticky mass is obtained.

The mixture of K₂DNABT-G thus includes the following components in the following amounts: K₂DNABT: 85.50 wt.-%; graphite: 9.50 wt.-%; and Nitrocellulose NC: 5.00 wt.-%.

The method described above is varied with the addition of half of the NC-solution (1234.5 mg) in order to achieve a reduced binder content of 5 wt.-%. The obtained suspension is dried using a nitrogen gas stream until the mixture became viscous.

In this example, an optimal viscosity, for the application of the mixture is not achieved due to the reduced binder content. The degree of “polymer-swelling” is lower and the mixture is more difficult to handle.

### Loading of Igniters

In preparation of the ignition pill, a syringe or pipette is used to apply the sticky mass of K₂DNABT-G to a series of the electronic igniters as shown in FIG. 1. After drying for 20 minutes, the loaded igniters are ready to test fire.

The loaded igniters are then connected to a power supply (U=20.5 V) with an interposed capacitor. The capacitor is charged by the power supply and the corresponding energy (E=½%(CU²)) is discharged by a fire button causing an electric current in the detonator chip.

For this test, five igniters were prepared with a mixture of 10 wt.-% binder and five with a mixture of 5 wt.-% binder. Both mixtures had a 100% firing rate.

1. A method of producing K₂DNABT which includes the steps of:
(a) reacting dialkyl carbonate with hydrazine hydrate to produce alkyl carbazate;
(b) reacting the alkyl carbazate with glyoxal to produce dialkyloxy carbonyl glyoxal bishydrazone;
(c) halogenating the dialkyloxy carbonyl glyoxal bishydrazone with a halogenating agent to form halogenated bishydrazone;
(d) azidation of the halogenated bishydrazone with an azide to produce diazido dialkyloxycarbonylglyoxal bishydrazone;
(e) cyclization of the diazido dialkyloxycarbonylglyoxal bishydrazone with a ring closing electrophile reactant to produce bistetrazole intermediate;
(f) deprotecting the bistetrazole intermediate with a nitrating agent to produce nitramino intermediate; and
(g) alkaline hydrolysing the nitramino intermediate with a potassium hydroxide to produce K₂DNABT;
wherein the nitrating agent is selected from the following: a mixture of about 10:1 nitric acid and phosphorous pentoxide; and a mixture of nitric acid with acetic anhydride in a range between 1:1 and 4:1.

2. (canceled)

3. A method according to claim 1 wherein the nitrating agent is the 4:1 mixture of nitric acid with acetic anhydride.

4. A method according to claim 1 or claim 3 wherein steps (a) and (b) are combined in a first one-pot reaction step in which hydrazine hydrate is added to dialkyl carbonate to form a alkyl carbazate intermediate, and then glyoxal is added to produce a dialkyloxy carbonyl glyoxal bishydrazone.

5. A method according to claim 1 or claim 3 wherein steps (c) and (d) are combined in a second one-pot reaction step in which dialkyloxy carbonyl glyoxal bishydrazone is dissolved in a first solvent before the halogenating (step (c)) to produce a halogenated bishydrazone intermediate and the azidation (step (d)) to produce diazido dialkyloxycarbonylglyoxal bishydrazone.

6. A method according to claim 1 or claim 3 wherein steps (d) and (e) are combined in a second one-pot reaction step in which the halogenated bishydrazone is dissolved in a second solvent before the azidation (step (d)) to form diazido dialkyloxycarbonylalyoxal bishydrazone and the cyclization (step (e)) to form the bistetrazole intermediate.

7. A method according to claim 5 wherein the diazido dialkvloxycarbonylglyoxal bishydrazone is dissolved in a second solvent before cyclization to produce the bistetrazole intermediate.

8. A method according to claim 5 wherein the first solvent is any of the following: DMF, DMSO, NMP, sulfolane, DMA, dioxane, water, EtOH, chloroform, MeOH, MeCN, THF, ethanol and water, and DMSO.

9. A method according to claim 1 or claim 3 wherein the halogenating agent is N-Chlorosuccinimide (NCS).

10. A method according to claim 1 or claim 3 wherein the azide is an earth metal azide.

11. A method according to claim 1 or claim 3 wherein the azide is sodium azide.

12. A method according to claim 7 wherein the second solvent is any of the following: DMF, ethanol, sulfolane, THF, MeCN, dioxane, chloroform.

13. A method according to claim 1 or claim 3 wherein the ring closing electrophile is selected from: HCl, SOCl₂, POCl₃, SO₂Cl₂, CO₂Cl₂, sulphuric acid and NaCl.

14. A method according to claim 13 wherein the electrophile is HCl in a 37% concentration.

15. A method according to claim 1 or claim 3 wherein the steps (f) and (g) are combined in a third one-pot reaction step in which the bistetrazole intermediate and the nitrating agent are added to produce a reaction mixture which is then directly added to a solution of potassium hydroxide to produce K₂DNABT.

16. A method according to claim 15 wherein the potassium hydroxide solution is a 85 wt. % solution.

17. (canceled)

18. (canceled)

19. A method according to claim 1 or claim 3 wherein the dialkyl carbonate is dimethyl carbonate or diethyl carbonate.

20. A method according to claim 19 wherein the dialkyl carbonate is diethyl carbonate.

21.-27. (canceled)

28. A method according to claim 4 wherein steps (c) and (d) are combined in a second one-pot reaction step in which dialkyloxy carbonyl glyoxal bishydrazone is dissolved in a first solvent before the halogenating (step (c)) to produce a halogenated bishydrazone intermediate and the azidation (step (d)) to produce diazido dialkyloxycarbonylglyoxal bishydrazone.

29. A method according to claim 5 wherein steps (d) and (e) are combined in a second one-pot reaction step in which the halogenated bishydrazone is dissolved in a second olvent before the azidation (step (d)) to form diazido dialkyloxycarbonylglyoxal bishydrazone and the cyclization (step (e)) to form the bistetrazole intermediate.
